SEQUENCE LISTING (8 sequences)

SEQ ID NO: 1 - protein, 8 aa, Artificial Sequence
  Synthetic Peptide
EIHGSLFK

SEQ ID NO: 2 - protein, 6 aa, Artificial Sequence
  Synthetic Peptide
HGSLFK

SEQ ID NO: 3 - protein, 8 aa, Artificial Sequence
  Synthetic Peptide
LVGAGVSA

SEQ ID NO: 4 - protein, 7 aa, Artificial Sequence
  Synthetic Peptide
GAGVSAE

SEQ ID NO: 5 - protein, 7 aa, Artificial Sequence
  Synthetic Peptide
GAGVSAE

SEQ ID NO: 6 - protein, 7 aa, Artificial Sequence
  Synthetic Peptide
TQNIDEL

SEQ ID NO: 7 - protein, 7 aa, Artificial Sequence
  Synthetic Peptide
TQNIDEL

SEQ ID NO: 8 - protein, 8 aa, Artificial Sequence
  Synthetic Peptide
VITQNIDA

(19) United States
(12) Patent Application Publication
(10) Pub. No.: US 2016/0354303 A1
(43) Pub. Date: Dec. 8, 2016

(54) Method, Compositions, And Kit For Modulating The Appearance Of Volume On Keratin Surfaces

(71) Applicant: ELC MANAGEMENT LLC, Melville, NY (US)

(72) Inventors: Nadine A. Pernodet, Huntington Station, NY (US); Daniel B. Yarosh, Merrick, NY (US); Donald F. Collins, Plainview, NY (US); James T. McCarthy, Babylon, NY (US)

(21) Appl. No.: 15/112,282

(22) PCT Filed: Feb. 6, 2015
(86) PCT No.: PCT/US2015/014900

§ 371 Date: Jul. 18, 2016

Related U.S. Application Data
(60) Provisional application No. 61/938,261, filed on Feb. 11, 2014.

(51) Int. Cl. A61K 8/97; A61Q 19/00; A61K 8/46; A61K 8/36; A61K 8/34; A61K 8/49

(52) U.S. Cl. CPC A61K 8/97; A61K 8/347; A61K 8/4926; A61K 8/46; A61K 8/361; A61Q 19/00

(57) ABSTRACT

A regimen and kit for treating select facial or body surfaces to modulate the appearance of the desired volume on the treated surface.

### CROSS-REFERENCE TO RELATED APPLICATIONS

This application claims priority of U.S. Provisional Patent Application Ser. No. 61/938,261 filed on Feb. 11, 2014.

### TECHNICAL FIELD

The invention is in the field of methods, compositions and treatment regimens for modulating the appearance of volume on the face and body.

### BACKGROUND OF THE INVENTION

Facial morphology, or the form and structure of the face, is a significant factor in the determination of what is a beautiful face versus what is not. Facial morphology depends on the form and shape of the skull and the underlying fat and muscle tissue. It is well known that aging affects facial morphology. The “baby fat” found in the faces of youth disappears with age. There is also a wasting of facial muscle mass with age, and it is known that the skull itself loses bony mass. The skin then becomes too large for the face. This in turn causes skin laxity, sagging and wrinkling.

Options for those who are interested in adding to, or redistributing, volume on the face often involve invasive surgical or dermatological procedures such as implants, injectable fillers, or fat transfer to the face from other areas of the body. Liposuction is one option for reducing volume on the body, but it is not used on the face due to potential for scarring.

Ways to affect facial morphology without invasive procedures are limited. Facial exercise is one known way that is said to be effective in adding facial volume. Facial exercise can be either manual or by using various commercially available devices that stimulate facial muscles electronically. Massage is also said to be effective in increasing the appearance of facial volume. More recently, acupuncture has been said to provide volumizing and wrinkle reduction through mechanisms which have yet to be completely understood.

However, to date, topically applied products have largely been ineffective or have effects of short duration. While some topically applied products may provide a short-lived appearance of volumizing, they do not have a long term effect. One example of transient volumizing effects can be found with lip products that contain ingredients that slightly irritate lips and cause them to temporarily swell with an effect that quickly dissipates.

Accordingly there is a need for compositions, treatment regimen and kit to affect facial morphology that causes a semi-permanent to permanent increase or decrease in volume instantaneously or over time when skin is topically treated.

### DESCRIPTION OF THE DRAWINGS

FIG. 1: shows the adipogenesis activity of Commipheroline.

FIG. 2: shows the decrease in human adipocyte generation after treatment with resveratrol.

FIG. 3: shows the adipocyte differentiation activity of CLA.

### SUMMARY OF THE INVENTION

A regimen for modulating the appearance of volume on select facial or body surfaces in need thereof comprising:
(a) identifying the surfaces for which modulating by increasing the appearance of volume is desired;
(b) treating the surface of (a), if identified, with an agent that causes the appearance of increased volume to the area of treatment;
(c) identifying the surfaces for which modulating by decreasing the appearance of volume is desired; and
(d) treating the surface of (c), if identified, with an agent that causes the appearance of decreased volume to the area of treatment.

The invention is also directed to a kit for modulating the appearance of volume on select facial or body surfaces in need thereof comprising:
(a) at least one agent for increasing the appearance of volume when used to treat a selected keratin surface;
(b) at least one agent for decreasing the appearance of volume when used to treat a selected keratin surface.

### DETAILED DESCRIPTION

All percentages used herein are percentages by weight unless otherwise indicated.

The term “modulating” means to change the appearance by either increasing or decreasing the appearance of volume on the surface that is modulated.

All patents and patent applications referred to by number herein are incorporated by reference in their entirety.

In the methods and regimens taught herein, it may be desired to treat only one select area of skin for either increase or decrease in appearance of volume. Accordingly, the regimen may include only one such desired treatment.

### I. The Volume Increase Agent

The agent for increasing the appearance of volume may be in the form of a composition, or a process that effectuates manual manipulation, such as what is achieved with a device, massage, or electronic muscle stimulators.

A. The Volume Increase Active

When the volume increase agent is an active ingredient of composition it will contain at least one such ingredient effective to cause the appearance of increased volume when topically applied. Such ingredient may be present in amounts ranging from about 0.001 to 10%, preferably from about 0.1 to 5%, more preferably from about 0.5 to 3% of the composition. The volume increase ingredient may cause the appearance of volume increase in a number of different biological pathways, including but not limited to topically applying actives that cause the appearance of volume to increase by stimulating adipogenesis or lipogenesis to stimulate fat cell production; stimulating the activity of various sirtuins such as SIRT1, 3, and 6 in fibroblasts which in turn stimulates synthesis of collagen or elastin, hyaluronic acid or filaggrin; or ingredients that cause skin to “plump”, such as humectants, polymers and other actives that firm and tone skin.

1. Ingredients that Stimulate Adipogenesis

Actives that stimulate adipogenesis may provide the appearance of increased volumization. Examples of adipogenesis stimulators include, but are not limited to, Commipheroline (Commiphora mukul extract); extract of Anemarrhena asphodeloides extract having a high content of Sarsasapogenin sold by Sederma under the trademark Volufiline®; Adansonia digitata pulp extract; Hibiscus sabdariffa flower extract, or a mixture of the two sold by Alban Muller under the Repulpami® trademark; Macadamia Ternifolia Seed Oil, sold in a mixture with tocopherol under the trademark Voluplus® by Centerchem; Sargaquinoic acid (SQA) and sargahydroquinoic acid (SHQA) from Sargassum yezoense extract; medium chain (C6-12) fatty carboxylic acids, preferably C6-12 octanoate or decanoate fatty acids; Acetyl hexapeptide-38, an ingredient sold by Lipotec under the trademark Adifyline®; Inonotus Obliquus (Chaga mushroom) extract; Bixa orellana extract (from Achiote plant); beta glucan; an ingredient sold under the trademark Adipofil®, which is a mixture of water, propanediol, omithine phospholipids, glycolipids; algae extract; extracts from date; and mixtures thereof.

2. Sirtuin Activators

Other ingredients that will provide the appearance of volume when used over time act by stimulating synthesis of collagen or elastin in fibroblasts including but not limited to ingredients that stimulate sirtuins such as SIRT1, 3, and 6.

Extracts from the Laminaria genus are known to be sirtuin activators. Laminaria is a genus that contains 30+ species of the brown algae Phaeophyceae, often referred to as kelp. Such extracts from the Laminaria genus include those of species abyssalis, agardhii, appressirhiza, brasiliensis, brongardiana, bulbosa, bullata, complanata, digitata, ephemera, farlowii, groenlandica, hyperborea, inclinitorhiza, multiplicata, nigripes, ochroleuca, pallida, platymeris, rodriguezi, ruprechtii, sachalinensis, setchellii, sinclairii, solidungula, or yezoensis. Preferred is where the extract from the Laminaria genus is also a SIRT3 activator. Preferred is where the extract is from Laminaria digitata, and more specifically an extract having laminarin content and/or a mannitol content of 1% by weight or greater, preferably around 2%. An example of a suitable extract of Laminaria digitata may be purchased from Barnet Products/Codif under the tradename Mitostime Di which is a mixture of 91 parts water, 8 parts Laminaria digitata extract, and 1 part preservative. Preferably the Laminaria digitata extract is obtained by aqueous extraction and leaching of lyophilized algae and sterilizing the microfiltration, followed by reverse osmosis to concentrate the active molecules. This extract is known to be a SIRT3 activator.

Also suitable as sirtuin activators are extracts from the Narcissus genus. Preferably the extract is a SIRT1 activator. Suggested ranges are from about 0.001 to 5%, preferably from about 0.01-4%, more preferably from about 0.05 to 1.5%. Examples of extracts include those from species alcaracensis, assoanus, asturiensis, bugei, bulbocodium, cyclamineus, jonquilla, longispathis, papyraceus, poeticus, pseudonarcissus, radingnaorum, romeiuxii, tazetta, triandrus, or medioluteus. More preferred is an extract from Narcissus tazetta, and in particular, an extract from the bulb when it is in the dormant state. Most preferred is Narcissus tazetta bulb extract purchased from IBR Dormin which is a mixture of about 62.5 parts glycerin, 37 parts water, and 0.5 parts Narcisuss tazetta bulb extract. The extract may be prepared as set forth in U.S. Pat. No. 6,635,287, hereby incorporated by reference in its entirety. In particular, the extract may be prepared by inducing dormancy in Narcissus bulbs by storing at temperatures around 45° C. for 2-24 hours. Water soluble extracts are then prepared by washing and disinfecting the bulbs, then cutting and homogenizing in distilled water. The resulting mass is then filtered to provide a liquid filtrate that contains the extract. In a most preferred embodiment of the invention the Narcissus extract is an activator of SIRT1 in fibroblasts.

Resveratrol derivatives such as those set forth in U.S. Pat. No. 8,084,496 are also suitable sirtuin activators. Examples include resveratrol ferulate, resveratrol salicylate, resveratrol ascorbate, and those resveratrol derivatives set forth in U.S. Pat. No. 8,084,496, hereby incorporated by reference in its entirety.

Also suitable are sirtuin activating peptides, such as those taught in U.S. Patent Application No. 2011/0318284 which is hereby incorporated by reference in its entirety. Such peptides have the general formula:

R₁-(AA)ₙ-X₁—X₂—X₃—X₄—X₅—X₆-(AA)ₚ-R₂ (I)

in which, X₁ is glycine or threonine or histidine;

X₂ is alanine or glutamine or glycine;

X₃ is glycine or asparagine or serine;

X₄ is valine or isoleucine or leucine;

X₅ is serine or aspartic acid or phenylalanine;

X₆ is alanine or glutamic acid or lysine;

and when X₁ is glycine then X₂ is alanine and X₃ is glycine;

when X₁ is threonine then X₃ is asparagine;

when X₁ is histidine then X₂ is glycine;

AA represents any amino acid and n and p are integers between 0 and 2;

R₁ represents the primary amino function of the N-terminal amino acid, free or substituted by an acyl type group having either an alkyl chain from C₁₋₃₀, saturated or unsaturated, that may be an acetyl group, or an aromatic group that may be chosen from among a benzoyl, tosyl or benzyloxycarbonyl type group; and

R₂ represents the hydroxyl group of the carboxyl function of the C-terminal amino acid, free or substituted by a group that may be chosen from among an alkyl chain from C1-30, or an NH₂, NHY or NYY group with Y representing an alkyl chain from C1-4.

More preferred are peptides corresponding to the following:

(SEQ ID No. 1):
Glu-Ile-His-Gly-Ser-Leu-Phe-Lys-NH₂
(SEQ ID No. 2)
His-Gly-Ser-Leu-Phe-Lys-NH₂
(SEQ ID No. 3)
Leu-Val-Gly-Ala-Gly-Val-Ser-Ala-NH₂
(SEQ ID No. 4)
Gly-Ala-Gly-Val-Ser-Ala-Glu
(SEQ ID No. 5)
Gly-Ala-Gly-Val-Ser-Ala-Glu-NH₂
(SEQ ID No. 6)
Thr-Gln-Asn-Ile-Asp-Glu-Leu
(SEQ ID No. 7)
Thr-Gln-Asn-Ile-Asp-Glu-Leu-NH₂
(SEQ ID No. 8)
Val-Ile-Thr-Gln-Asn-Ile-Asp-Ala-NH₂

In one preferred embodiment, the peptides of SEQ ID No. 5 and 7 are preferred.

The SIRT6 activating peptide may be present in the composition in amounts ranging from 0.0001 to 8%, preferably from about 0.001 to 3%, more preferably from about 0.01 to 1%. The term “SIRT6 activating peptide” means a peptide that causes the amount of SIRT6 in the cell to increase by whatever pathway causes that result, and where the types of cells include keratinocytes, dermal fibroblasts, etc. Most preferred is the peptide having SEQ ID No. 5.

In another embodiment, the peptide may be found as a component of an extract from yeast. It has also been shown to activate SIRT6. If present, the may be present in the yeast extract composition in amounts ranging from 10 to 1,000,000 ppm, or from 100 to 100,000 ppm, or even from 1,000 to 10,000 ppm.

3. Hydrogels

Hydrogels may also be effective for providing the appearance of volume on topically applied areas. Examples of such hydrogels include, but are not limited to, those which are known as hyaluronic acid based dermal fillers, which may also be suitable for topical application. For example hyaluronic acid that is crosslinked with various suitable crosslinking agents including alpha omega dienes, or diamine or multi-amine crosslinking agents, hexamethylene diamine, is most suitable for topical application. The reaction of hyaluronic acid and the amine crosslinking agents may be conducted in the presence of a carbodiimide. A particularly suitable polymer is a glycosaminoglycan. Suitable hydrogels are described in PCT/US/2012067993.

4. Skin Batteries

Another suitable way to increase the appearance of volume is with “skin batteries”, which are patches or compositions that facilitate the running of a small current via electrical potential through the skin. An example of such skin batteries are set forth in U.S. Pat. Nos. 6,306,384 and 6,033,655.

One preferred route is by use of magnetic particles bound into ion exchange resins, as set forth in U.S. Pat. No. 6,033,655. A magnetic based slurry is formed by combining iron based magnetic particles with water (e.g. 500 g H₂O is added 5 g FeCl₂H₂O and the mixture filtered to remove resulting debris. After filtration the mixture is blended with a suitable ingredient (such as 10 g Sephadex SP-C50) to form a slurry. The slurry is then added to sodium hydroxide solution (500 g 5% NaOH solution) and washed and filtered several times. In the resulting product the magnetic iron particles are bound to the Sephadex beads and are ready for incorporation into a composition for treating select areas of the skin. The resulting product is again filtered, then washed and filtered several times to remove any residual hydroxide, producing a product in which Fe(OH).sub.2 is bound to Sephadex beads.

The particle composition, as a slurry, is allowed to stand at room temperature, typically for about one hour, to accomplish air oxidation. The slurry is then weighed on a standard scale and rinsed with an equal amount of 0.25% triethanolamine to achieve a pH of about 9. At this point, they are ready to be incorporated into a product.

Also suitable are compositions or agents which have both electronic donor and electron receiving capability as discussed in U.S. Pat. No. 6,306,384. Like a traditional battery, one component of the two part composition acts as a negative electrode that releases electrons into an external circuit, a second component acts as a positive electrode that gains electrons from an external circuit. Each component is maintained in a matrix which is capable of acting as an electrolyte to facilitate the electron transfer between anode and cathode.

Like a traditional battery, the active materials in the two components are selected for their ability to, respectively, either donate or accept electrons. In general, the first electrode is usually a metallic element, or an oxide or salt thereof, with a positive oxidation potential, that is, capable of giving up electrons. Examples of such metallic elements include, but are not limited to, copper or iron. The second electrode is often a metal oxide, hydroxide, halide or sulfide, for example, of gold, silver, platinum or palladium, or appropriate ions thereof. Iron may actually act as either electrode depending on the identity of the other electrode. The term “effective amount” refers to the amount of one electrode component which is sufficient to generate a measurable potential on the skin when combined with an effective amount of a properly selected opposite electrode component. In a typical composition, as applied on the skin, the individual electrode components are preferably present in an amount of from 0.0001-20% by weight of the formulation. It is possible for the two electrode components can be combined in the same nonconductive carrier, in a single nonconductive container, and no premature generation of current will occur. The skin itself, being conductive, can provide the necessary electrolyte function, and the single carrier containing both electrodes can be applied simultaneously and directly to the skin, thereby generating the potential once it is in contact with the skin.

Also suitable as volume increase agents are “body corrective” formulations as set forth in PCT/US/2011/050003 (WO/2012/030984). These formulations are silicones that are crosslinked on the skin surface to provide the desired morphological change.

B. The Volume Increase Process

The volume increase agent may be in the form of physical manipulation of the skin or body surface, including by massage, either digitally or with devices, or through electrical muscle stimulation. These types of manipulation will stimulate the underlying muscle and tissue and cause the appearance of volume increase. Examples of hand facial or body massage are well known. Examples of devices that massage skin surfaces or perform electric muscle stimulation are also well known. Another suitable agent may be a massaging end piece for the face as set forth WO/2013/014099. Also suitable are pressure techniques such as massage using fingers and palms as disclosed in WO/2012/161455 where pressure and massage are combined, optionally with a cosmetic, and massaged into the skin to cause an appearance of increased volume.

Also suitable as agents for increasing the appearance of volume are transoral methods as disclosed in PCT/US2012/034424 (WO/2012/145609) where films or compositions are applied to the oral mucosa or teeth and stimulate the appearance of volume by infusing into oral mucosa.

C. The Volume Decrease Agent

When the volume decrease agent is a composition, the composition comprises at least one volume decrease ingredient. Such ingredient may be present in amounts ranging from about 0.001 to 10%, preferably from about 0.1 to 5%, more preferably from about 0.5 to 3% of the composition. The volume decrease ingredient may cause the appearance of volume decrease in a number of different ways, including but not limited to topically applying actives that cause the appearance of volume to decrease by stimulating lipolysiss; tightening skin, or drawing water from the skin.

1. Adipogenesis Inhibitors or Lipolytic Agents

Suitable volume decrease agents include ingredients that inhibit adipogenesis or stimulate lipolysis of adipocytes. Examples of such ingredients include conjugated linoleic acid, forskolin, resveratrol, esters of resveratrol and inorganic acids such as phosphoric, sulfuric, hydrochloric acids and the like; hydrolyzed Myrtus Communis leaf extract (sold under the trademark Longevicell® by Silab), and the like. Particularly suitable are trisodium resveratrol triphosphate, resveratrol, and Myrtus Communis leaf extract. A variety of other ingredients may also be suitable including but not limited to COX-2 (cyclooxygenase-2) inhibitors (such as extracts of plants from the Acacia genus, Calluna Vulgaris, Rosa Canina fruit extract, nettle leaf extract, Polyporus Umbellatus, Chamomilla Recutita (Matricaria) flower oil, Coleus Forskohlii extract, Rusperin C® (a mixture of Butcher's Broom extract and heperidin methyl chalcone), HOP's beta acids, and mixtures thereof); TRB3 or Tribble 3, a kinase; calcineurin; C75, a fatty acid synthase inhibitor, tripeptides of the sequence Val-Tyr-Pro or Val-Thr-Leu; A-type lamnins; various types of calpain inhibitors; mifepristone; protein kinase C activators; lipooxygenase inhibitors such as Haplophyllum hispanicum Spach, Scutellaria Rivularis extracts and its active components, alpha ketoboswellic acid, Zileuton® (1-(1-benzothiophen-2-ylethyl)-1-hydroxy-urea), Centaurium extract, nordihydroguaiaretic acid, ginger oil, caffeine, niacinamide, resveratrol, retinol, Polygonum Cuspidatum extract, cedar himalaya extract, tetrahydrocurcuminoids, Nigella Sativa essential oil, copaiba balsam, botulism toxin, Australian sandalwood, Rhodiola Rosea, frankincense, blue cypress oil, blue chamomile oil, anti-inflammatory oil blend, vetiver mada, bulgarian rose oil, resveratrol ferulate, Helichrysum oil, rosmarinic acid, resveratrol triphosphate, noni leaf extract; stromelysin-3; endrin; glucocorticoids; Caralluma Fimbriata Extract, Mucuna Extract, Bacana Extract, G-alpha 13 inhibitors such as those disclosed WO/2003/10449, hereby incorporated by references in its entirety. Such G-alpha 13 inhibitors include antibodies, peptides (including dominant negative peptides) and antisense compounds, including ribozymes, inhibitory RNA molecules including siRNA molecules and antisense oligonucleotides; and so on.

Suitable adipocyte lipolytic agents may be identified by conducting a simple test using a lipolysis assay kit purchased from Zen-Bio. One suitable kit is the Lypolysis Assay Kit (dual glycerol and free fatty acids detection, reagents+cells) purchased from Zen-Bio (www.zen-bio.com) under catalog no. LIP-3.

Suitable lipolytic agents include conjugated linoleic acid; forskolin. Other ingredients are known to be lipolytic agents as well, such as nicotine; deoxycholate; theophylline; caffeine; theobromine; magnolol (a fraction of Magnolia extract); isoproterenol; kaempferol-7-O-Neohesperidoside; Clary extract; Chrystanthellum extract, specifically Chrysanthellum Indicum; and the like.

2. Skin Tightening Agents

The volume decrease agent may also be in the form of a skin tightening agent. Suitable skin tightening agents include ingredients that have a contractile effect when applied to the skin, such as silicone elastomers, various polymers or copolymers, and the like.

Polymeric or copolymeric ingredients include homo- or copolymers of acrylates, methacrylates, acrylamides, methacrylamides, urethanes, and the like, which may be copolymerized or derivatized with long chain alkyl, ether, or alkoxy groups. Examples include acrylamides copolymer, acrylamide/sodium acrylate copolymer, ammonium acryloyldimethyl taurate, ammonium acryloyldimethyltaurate/VP copolymer, acrylates C10-30 alkyl acrylate crosspolymer, acrylates/beheneth-25 methacrylate copolymer, acrylates/vinyl neodecanoate copolymer, acrylates ceteth-20 itaconate copolymer, acrylates ceteth-20 methacrylate copolymer, acrylates steareth-20 methacrylate copolymer, acrylates steareth-50 methacrylate copolymer, ammonium acryloyldimethyltaurate/steareth-8 methacrylate copolymer, acrylates/palmeth-25 itaconate copolymer, acrylates/palmeth-25 acrylate copolymer, and the like.

Also suitable as skin tightening agents are various types of silicone elastomers, that is crosslinked silicones formed by the reaction of vinyl-terminated siloxanes and methylhydroxydimethyl siloxanes. Examples of such silicone elastomers include those sold under the trademarks Gransil™ by Grant Industries, Botanisil™ by Botanigenics, Actiprime™ by Active Organics, KSG or USG by Shin-Etsu Silicones; or 9040, 9044 silicone elastomers or blends thereof by Dow Corning Corporation. Such elastomers include but are not limited to cetearyl dimethicone/vinyl dimethicone crosspolymer, dimethicone/vinyl dimethicone crosspolymer, stearyl dimethicone/vinyl dimethicone crosspolymer, cetyl dimethicone bis-vinyl dimethicone crosspolymer, dimethicone crosspolymer-3, dimethicone/divinyl dimethicone/silsesquioxane crosspolymer, diphenyl dimethicone/vinyl dimethicone crosspolymer, polydimethylsiloxyethyldimethicone/bis-vinyl dimethicone crosspolymer, and so on.

Other suitable ingredients for use in formulating the treatment agent when in the form of a composition include, but are not limited to those further set forth herein.

D. Other Ingredients

1. Botanical Extracts

It may be desirable to include one or more botanical extracts in the compositions. If so, suggested ranges are from about 0.0001 to 10%, preferably about 0.0005 to 8%, more preferably about 0.001 to 5% by weight of the total composition. Suitable botanical extracts include extracts from plants (herbs, roots, flowers, fruits, seeds) such as flowers, fruits, vegetables, and so on, including yeast ferment extract, Padina Pavonica extract, thermus thermophilis ferment extract, camelina sativa seed oil, boswellia serrata extract, olive extract, Aribodopsis Thaliana extract, Acacia Dealbata extract, Acer Saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, caffeine, green tea, chamomile, willowbark, mulberry, poppy, and those set forth on pages 1646 through 1660 of the CTFA Cosmetic Ingredient Handbook, Eighth Edition, Volume 2. Further specific examples include, but are not limited to, Glycyrrhiza Glabra, Salix Nigra, Macrocycstis Pyrifera, Pyrus Malus, Saxifraga Sarmentosa, Vitis Vinifera, Morus Nigra, Scutellaria Baicalensis, Anthemis Nobilis, Salvia Sclarea, Rosmarinus Officianalis, Citrus Medica Limonum, Panax Ginseng, Siegesbeckia Orientalis, Fructus Mume, Ascophyllum Nodosum, Bifida Ferment lysate, Glycine Soja extract, Beta Vulgaris, Haberlea Rhodopensis, Polygonum Cuspidatum, Citrus Aurantium Dulcis, Vitis Vinifera, Selaginella Tamariscina, Humulus Lupulus, Citrus Reticulata Peel, Punica Granatum, Asparagopsis, Curcuma Longa, Menyanthes Trifoliata, Helianthus Annuus, Hordeum Vulgare, Cucumis Sativus, Evernia Prunastri, Evernia Furfuracea, and mixtures thereof.

2. Oils

In the event the compositions of the invention are in emulsion form, the composition may comprise an oil phase. If present, suggested amounts range from 0.1 to 80%, preferably from 0.5 to 75%, more preferably from 1 to 50%. Suitable oils include silicones, esters, vegetable oils, synthetic oils, including but not limited to those set forth herein. The oils may be volatile or nonvolatile, and are preferably in the form of a pourable liquid at room temperature. The term “volatile” means that the oil has a measurable vapor pressure, or a vapor pressure of at least about 2 mm. of mercury at 20° C. The term “nonvolatile” means that the oil has a vapor pressure of less than about 2 mm. of mercury at 20° C.

(a). Volatile Oils

Suitable volatile oils generally have a viscosity ranging from about 0.5 to 5 centistokes 25° C. and include linear silicones, cyclic silicones, paraffinic hydrocarbons, or mixtures thereof. Volatile oils may be used to promote more rapid drying of the skin care composition after it is applied to skin.

(i). Volatile Silicones

Cyclic silicones are one type of volatile silicone that may be used in the composition. Such silicones have the general formula:

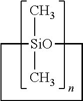

where n=3-6, preferably 4, 5, or 6.

Also suitable are linear volatile silicones, for example, those having the general formula:

(CH₃)₃Si—O—[Si(CH₃)₂—O]ₙ—Si(CH₃)₃

where n=0, 1, 2, 3, 4, or 5, preferably 0, 1, 2, 3, or 4.

Cyclic and linear volatile silicones are available from various commercial sources including Dow Corning Corporation and Momentive Performance Materials. The Dow Corning linear volatile silicones are sold under the tradenames Dow Corning 244, 245, 344, and 200 fluids. These fluids include hexamethyldisiloxane (viscosity 0.65 centistokes (abbreviated cst)), octamethyltrisiloxane (1.0 cst), decamethyltetrasiloxane (1.5 cst), dodecamethylpentasiloxane (2 cst) and mixtures thereof, with all viscosity measurements being at 25° C.

Suitable branched volatile silicones include alkyl trimethicones such as methyl trimethicone, a branched volatile silicone having the general formula:

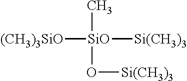

Methyl trimethicone may be purchased from Shin-Etsu Silicones under the tradename TMF-1.5, having a viscosity of 1.5 centistokes at 25° C.

(ii). Volatile Paraffinic Hydrocarbons

Also suitable as the volatile oils are various straight or branched chain paraffinic hydrocarbons having 5, 6, 7, 8, 9, 10, 11, 12, 13, 14, 15, 16, 17, 18, 19, or 20 carbon atoms, more preferably 8 to 16 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference.

Preferred volatile paraffinic hydrocarbons have a molecular weight of 70-225, preferably 160 to 190 and a boiling point range of 30 to 320, preferably 60 to 260° C., and a viscosity of less than about 10 cst. at 25° C. Such paraffinic hydrocarbons are available from EXXON under the ISOPARS trademark, and from the Permethyl Corporation and include isododecane and isohexadecane.

(b). Non-Volatile Oils

A variety of nonvolatile oils are also suitable for use in the compositions of the invention. The nonvolatile oils generally have a viscosity of greater than about 5 to 10 centistokes at 25° C., and may range in viscosity up to about 1,000,000 centipoise at 25° C. Examples of nonvolatile oils include, but are not limited to:

(i). Esters

Suitable esters are mono-, di-, and triesters. The composition may comprise one or more esters selected from the group, or mixtures thereof.

### (aa). Monoesters

Monoesters are defined as esters formed by the reaction of a monocarboxylic acid having the formula R—COOH, wherein R is a straight or branched chain saturated or unsaturated alkyl having 2 to 45 carbon atoms, or phenyl; and an alcohol having the formula R—OH wherein R is a straight or branched chain saturated or unsaturated alkyl having 2-30 carbon atoms, or phenyl. Both the alcohol and the acid may be substituted with one or more hydroxyl groups. Either one or both of the acid or alcohol may be a “fatty” acid or alcohol, and may have from about 6 to 30 carbon atoms, more preferably 12, 14, 16, 18, or 22 carbon atoms in straight or branched chain, saturated or unsaturated form. Examples of monoester oils that may be used in the compositions of the invention include hexyl laurate, butyl isostearate, hexadecyl isostearate, cetyl palmitate, isostearyl neopentanoate, stearyl heptanoate, isostearyl isononanoate, steary lactate, stearyl octanoate, stearyl stearate, isononyl isononanoate, and so on.

### (bb). Diesters

Suitable diesters are the reaction product of a dicarboxylic acid and an aliphatic or aromatic alcohol or an aliphatic or aromatic alcohol having at least two substituted hydroxyl groups and a monocarboxylic acid. The dicarboxylic acid may contain from 2 to 30 carbon atoms, and may be in the straight or branched chain, saturated or unsaturated form. The dicarboxylic acid may be substituted with one or more hydroxyl groups. The aliphatic or aromatic alcohol may also contain 2 to 30 carbon atoms, and may be in the straight or branched chain, saturated, or unsaturated form. Preferably, one or more of the acid or alcohol is a fatty acid or alcohol, i.e. contains 12-22 carbon atoms. The dicarboxylic acid may also be an alpha hydroxy acid. The ester may be in the dimer or trimer form. Examples of diester oils that may be used in the compositions of the invention include diisotearyl malate, neopentyl glycol dioctanoate, dibutyl sebacate, dicetearyl dimer dilinoleate, dicetyl adipate, diisocetyl adipate, diisononyl adipate, diisostearyl dimer dilinoleate, diisostearyl fumarate, diisostearyl malate, dioctyl malate, and so on.

### (cc). Triesters

Suitable triesters comprise the reaction product of a tricarboxylic acid and an aliphatic or aromatic alcohol or alternatively the reaction product of an aliphatic or aromatic alcohol having three or more substituted hydroxyl groups with a monocarboxylic acid. As with the mono- and diesters mentioned above, the acid and alcohol contain 2 to 30 carbon atoms, and may be saturated or unsaturated, straight or branched chain, and may be substituted with one or more hydroxyl groups. Preferably, one or more of the acid or alcohol is a fatty acid or alcohol containing 12 to 22 carbon atoms. Examples of triesters include esters of arachidonic, citric, or behenic acids, such as triarachidin, tributyl citrate, triisostearyl citrate, tri C₁₂₋₁₃ alkyl citrate, tricaprylin, tricaprylyl citrate, tridecyl behenate, trioctyldodecyl citrate, tridecyl behenate; or tridecyl cocoate, tridecyl isononanoate, and so on.

Esters suitable for use in the composition are further described in the C.T.F.A. Cosmetic Ingredient Dictionary and Handbook, Eleventh Edition, 2006, under the classification of “Esters”, the text of which is hereby incorporated by reference in its entirety.

(ii). Hydrocarbon Oils

It may be desirable to incorporate one or more nonvolatile hydrocarbon oils into the composition. Suitable nonvolatile hydrocarbon oils include paraffinic hydrocarbons and olefins, preferably those having greater than about 20 carbon atoms. Examples of such hydrocarbon oils include C₂₄₋₂₈ olefins, C₃₀₋₄₅ olefins, C₂₀₋₄₀ isoparaffins, hydrogenated polyisobutene, polyisobutene, polydecene, hydrogenated polydecene, mineral oil, pentahydrosqualene, squalene, squalane, and mixtures thereof. In one preferred embodiment such hydrocarbons have a molecular weight ranging from about 300 to 1000 Daltons.

(iii). Glyceryl Esters of Fatty Acids

Synthetic or naturally occurring glyceryl esters of fatty acids, or triglycerides, are also suitable for use in the compositions. Both vegetable and animal sources may be used. Examples of such oils include castor oil, lanolin oil, C₁₀₋₁₈ triglycerides, caprylic/capric/triglycerides, sweet almond oil, apricot kernel oil, sesame oil, camelina sativa oil, tamanu seed oil, coconut oil, corn oil, cottonseed oil, linseed oil, ink oil, olive oil, palm oil, illipe butter, rapeseed oil, soybean oil, grapeseed oil, sunflower seed oil, walnut oil, and the like.

Also suitable are synthetic or semi-synthetic glyceryl esters, such as fatty acid mono-, di-, and triglycerides which are natural fats or oils that have been modified, for example, mono-, di- or triesters of polyols such as glycerin. In an example, a fatty (C₁₂₋₂₂) carboxylic acid is reacted with one or more repeating glyceryl groups. glyceryl stearate, diglyceryl diiosostearate, polyglyceryl-3 isostearate, polyglyceryl-4 isostearate, polyglyceryl-6 ricinoleate, glyceryl dioleate, glyceryl diisotearate, glyceryl tetraisostearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

(iv). Nonvolatile Silicones

Nonvolatile silicone oils, both water soluble and water insoluble, are also suitable for use in the composition. Such silicones preferably have a viscosity ranging from about greater than 5 to 800,000 cst, preferably 20 to 200,000 cst at 25° C. Suitable water insoluble silicones include amine functional silicones such as amodimethicone.

For example, such nonvolatile silicones may have the following general formula:

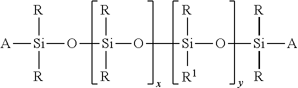

wherein R and R′ are each independently C₁₋₃₀ straight or branched chain, saturated or unsaturated alkyl, phenyl or aryl, trialkylsiloxy, and x and y are each independently 1-1,000,000; with the proviso that there is at least one of either x or y, and A is alkyl siloxy endcap unit. Preferred is where A is a methyl siloxy endcap unit; in particular trimethylsiloxy, and R and R′ are each independently a C₁₋₃₀ straight or branched chain alkyl, phenyl, or trimethylsiloxy, more preferably a C₁₋₂₂ alkyl, phenyl, or trimethylsiloxy, most preferably methyl, phenyl, or trimethylsiloxy, and resulting silicone is dimethicone, phenyl dimethicone, diphenyl dimethicone, phenyl trimethicone, or trimethylsiloxyphenyl dimethicone. Other examples include alkyl dimethicones such as cetyl dimethicone, and the like wherein at least one R is a fatty alkyl (C₁₂, C₁₄, C₁₆, C₁₈, C₂₀, or C₂₂), and the other R is methyl, and A is a trimethylsiloxy endcap unit, provided such alkyl dimethicone is a pourable liquid at room temperature. Phenyl trimethicone can be purchased from Dow Corning Corporation under the tradename 556 Fluid. Trimethylsiloxyphenyl dimethicone can be purchased from Wacker-Chemie under the tradename PDM-1000. Cetyl dimethicone, also referred to as a liquid silicone wax, may be purchased from Dow Corning as Fluid 2502, or from DeGussa Care & Surface Specialties under the trade names Abil Wax 9801, or 9814.

(v). Fluorinated Oils

Various types of fluorinated oils may also be suitable for use in the compositions including but not limited to fluorinated silicones, fluorinated esters, or perfluropolyethers. Particularly suitable are fluorosilicones such as trimethylsilyl endcapped fluorosilicone oil, polytrifluoropropylmethylsiloxanes, and similar silicones such as those disclosed in U.S. Pat. No. 5,118,496 which is hereby incorporated by reference. Perfluoropolyethers include those disclosed in U.S. Pat. Nos. 5,183,589, 4,803,067, 5,183,588 all of which are hereby incorporated by reference, which are commercially available from Montefluos under the trademark Fomblin.

3. Aqueous Phase Structuring Agents

In the case where the compositions are in the form of aqueous solutions, dispersions or emulsions, in addition to water the aqueous phase may contain one or more aqueous phase structuring agents, that is, an agent that increases the viscosity or, or thickens, the aqueous phase of the composition. This is particularly desirable when the composition is in the form of a serum or gel. Suitable ranges of aqueous phase structuring agent, if present, are from about 0.01 to 30%, preferably from about 0.1 to 20%, more preferably from about 0.5 to 15% by weight of the total composition. Examples of such agents include various acrylate based thickening agents, natural or synthetic gums, polysaccharides, and the like, including but not limited to those set forth below.

(a). Acrylate Polymers

Also suitable are different types of synthetic polymeric thickeners that are other than the polymers set forth above. If present, suggested amounts are from 0.1 to 40%, preferably from about 0.5 to 35%, more preferably from about 1 to 25%.

One type includes acrylic polymeric thickeners comprised of monomers A and B wherein A is selected from the group consisting of acrylic acid, methacrylic acid, and mixtures thereof; and B is selected from the group consisting of a C₁₋₂₂ alkyl acrylate, a C₁₋₂₂ alky methacrylate, and mixtures thereof are suitable. In one embodiment the A monomer comprises one or more of acrylic acid or methacrylic acid, and the B monomer is selected from the group consisting of a C₁₋₁₀, most preferably C₁₋₄ alkyl acrylate, a C₁₋₁₀, most preferably C₁₋₄ alkyl methacrylate, and mixtures thereof. Most preferably the B monomer is one or more of methyl or ethyl acrylate or methacrylate. The acrylic copolymer may be supplied in an aqueous solution having a solids content ranging from about 10-60%, preferably 20-50%, more preferably 25-45% by weight of the polymer, with the remainder water. The composition of the acrylic copolymer may contain from about 0.1-99 parts of the A monomer, and about 0.1-99 parts of the B monomer. Acrylic polymer solutions include those sold by Seppic, Inc., under the tradename Capigel.

Also suitable are acrylic polymeric thickeners that are copolymer of A, B, and C monomers wherein A and B are as defined above, and C has the general formula:

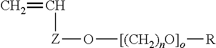

wherein Z is —(CH₂)ₘ; wherein m is 1-10, n is 2-3, o is 2-200, and R is a C₁₀₋₃₀ straight or branched chain alkyl. Examples of the secondary thickening agent above, are copolymers where A and B are defined as above, and C is CO, and wherein n, o, and R are as above defined. Examples of such secondary thickening agents include acrylates/steareth-20 methacrylate copolymer, which is sold by Rohm & Haas under the tradename Acrysol ICS-1.

Also suitable are acrylate based anionic amphiphilic polymers containing at least one hydrophilic unit and at least one allyl ether unit containing a fatty chain. Preferred are those where the hydrophilic unit contains an ethylenically unsaturated anionic monomer, more specifically a vinyl carboxylic acid such as acrylic acid, methacrylic acid or mixtures thereof, and where the allyl ether unit containing a fatty chain corresponds to the monomer of formula:

CH₂═CR′CH₂OBₙR

in which R′ denotes H or CH₃, B denotes the ethylenoxy radical, n is zero or an integer ranging from 1 to 100, R denotes a hydrocarbon radical selected from alkyl, arylalkyl, aryl, alkylaryl and cycloalkyl radicals which contain from 8 to 30 carbon atoms, preferably from 10 to 24, and even more particularly from 12 to 18 carbon atoms. More preferred in this case is where R′ denotes H, n is equal to 10 and R denotes a stearyl (C18) radical. Anionic amphiphilic polymers of this type are described and prepared in U.S. Pat. Nos. 4,677,152 and 4,702,844, both of which are hereby incorporated by reference in their entirety. Among these anionic amphiphilic polymers, polymers formed of 20 to 60% by weight acrylic acid and/or methacrylic acid, of 5 to 60% by weight lower alkyl methacrylates, of 2 to 50% by weight allyl ether containing a fatty chain as mentioned above, and of 0 to 1% by weight of a crosslinking agent which is a well-known copolymerizable polyethylenic unsaturated monomer, for instance diallyl phthalate, allyl (meth)acrylate, divinylbenzene, (poly)ethylene glycol dimethacrylate and methylenebisacrylamide. One commercial example of such polymers are crosslinked terpolymers of methacrylic acid, of ethyl acrylate, of polyethylene glycol (having 10 EO units) ether of stearyl alcohol or steareth-10, in particular those sold by the company Allied Colloids under the names SALCARE SC80 and SALCARE SC90, which are aqueous emulsions containing 30% of a crosslinked terpolymer of methacrylic acid, of ethyl acrylate and of steareth-10 allyl ether (40/50/10).

Also suitable are acrylate copolymers such as Polyacrylate-3 which is a copolymer of methacrylic acid, methylmethacrylate, methylstyrene isopropylisocyanate, and PEG-40 behenate monomers; Polyacrylate-10 which is a copolymer of sodium acryloyldimethyltaurate, sodium acrylate, acrylamide and vinyl pyrrolidone monomers; or Polyacrylate-11, which is a copolymer of sodium acryloyldimethylacryloyldimethyl taurate, sodium acrylate, hydroxyethyl acrylate, lauryl acrylate, butyl acrylate, and acrylamide monomers.

Also suitable are crosslinked acrylate based polymers where one or more of the acrylic groups may have substituted long chain alkyl (such as 6-40, 10-30, and the like) groups, for example acrylates/C₁₀₋₃₀ alkyl acrylate crosspolymer which is a copolymer of C₁₀₋₃₀ alkyl acrylate and one or more monomers of acrylic acid, methacrylic acid, or one of their simple esters crosslinked with the allyl ether of sucrose or the allyl ether of pentaerythritol. Such polymers are commonly sold under the Carbopol or Pemulen tradenames and have the CTFA name carbomer.

One particularly suitable type of aqueous phase thickening agent are acrylate based polymeric thickeners sold by Clariant under the Aristoflex trademark such as Aristoflex AVC, which is ammonium acryloyldimethyltaurate/VP copolymer, Aristoflex AVL which is the same polymer has found in AVC dispersed in mixture containing caprylic/capric triglyceride, trilaureth-4, and polyglyceryl-2 sesquiisostearate; or Aristoflex HMB which is ammonium acryloyldimethyltaurate/beheneth-25 methacrylate crosspolymer, and the like.

(b). High Molecular Weight PEG or Polyglycerins

Also suitable as the aqueous phase thickening agents are various polyethylene glycols (PEG) derivatives where the degree of polymerization ranges from 1,000 to 200,000. Such ingredients are indicated by the designation “PEG” followed by the degree of polymerization in thousands, such as PEG-45M, which means PEG having 45,000 repeating ethylene oxide units. Examples of suitable PEG derivatives include PEG 2M, 5M, 7M, 9M, 14M, 20M, 23M, 25M, 45M, 65M, 90M, 115M, 160M, 180M, and the like.

Also suitable are polyglycerins which are repeating glycerin moieties where the number of repeating moieties ranges from 15 to 200, preferably from about 20-100. Examples of suitable polyglycerins include those having the CFTA names polyglycerin-20, polyglycerin-40, and the like.

4. Oil Phase Structuring Agents

In the case where the composition is anhydrous or in the form of an emulsion, it may be desirable to include one or more oil phase structuring agents in the cosmetic composition. The term “oil phase structuring agent” means an ingredient or combination of ingredients, soluble or dispersible in the oil phase, which will increase the viscosity, or structure, the oil phase. The structuring agent may be present in an amount sufficient to provide a liquid composition with increased viscosity, a semi-solid, or in some cases a solid composition that may be self-supporting. The structuring agent itself may be present in the liquid, semi-solid, or solid form. Suggested ranges of structuring agent are from about 0.01 to 70%, preferably from about 0.05 to 50%, more preferably from about 0.1-35% by weight of the total composition. Suitable oil phase structuring agents include those that are silicone based or organic based. They may be polymers or non-polymers, synthetic, natural, or a combination of both.

(a). Silicone Structuring Agents

A variety of oil phase structuring agents may be silicone based, such as silicone gums, silicone waxes, linear silicones having a degree of polymerization that provides the silicone with a degree of viscosity such that when incorporated into the cosmetic composition it is capable of increasing the viscosity of the oil phase. Examples of silicone structuring agents include, but are not limited to:

(i). Silicone Gums

Also suitable for use as an oil phase structuring agent are one or more silicone gums. The term “gum” means a silicone polymer having a degree of polymerization sufficient to provide a silicone having a gum-like texture. In certain cases the silicone polymer forming the gum may be crosslinked. The silicone gum typically has a viscosity ranging from about 500,000 to 100 million cst at 25° C., preferably from about 600,000 to 20 million, more preferably from about 600,000 to 12 million cst. All ranges mentioned herein include all subranges, e.g. 550,000; 925,000; 3.5 million.

The silicone gums that are used in the compositions include, but are not limited to, those of the general formula wherein:

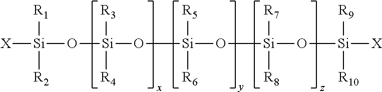

R₁ to R₉ are each independently an alkyl having 1 to 30 carbon atoms, aryl, or aralkyl; and X is OH or a C₁₋₃₀ alkyl, or vinyl; and wherein x, y, or z may be zero with the proviso that no more than two of x, y, or z are zero at any one time, and further that x, y, and z are such that the silicone gum has a viscosity of at least about 500,000 cst, ranging up to about 100 million centistokes at 25° C. Preferred is where R is methyl or OH.

Such silicone gums may be purchased in pure form from a variety of silicone manufacturers including Wacker-Chemie or Dow Corning, and the like. Such silicone gums include those sold by Wacker-Belsil under the trade names CM3092, Wacker-Belsil 1000, or Wacker-Belsil DM 3096. A silicone gum where X is OH, also referred to as dimethiconol, is available from Dow Corning Corporation under the trade name 1401. The silicone gum may also be purchased in the form of a solution or dispersion in a silicone compatible vehicle such as volatile or nonvolatile silicone. An example of such a mixture may be purchased from Barnet Silicones under the HL-88 tradename, having the INCI name dimethicone.

(ii). Silicone Waxes

Another type of oily phase structuring agent includes silicone waxes that are typically referred to as alkyl silicone waxes which are semi-solids or solids at room temperature. The term “alkyl silicone wax” means a polydimethylsiloxane having a substituted long chain alkyl (such as C16 to 30) that confers a semi-solid or solid property to the siloxane. Examples of such silicone waxes include stearyl dimethicone, which may be purchased from DeGussa Care & Surface Specialties under the tradename Abil Wax 9800 or from Dow Corning under the tradename 2503. Another example is bis-stearyl dimethicone, which may be purchased from Gransil Industries under the tradename Gransil A-18, or behenyl dimethicone, behenoxy dimethicone.

(iii). Polyamides or Silicone Polyamides

Also suitable as oil phase structuring agents are various types of polymeric compounds such as polyamides or silicone polyamides.

The term silicone polyamide means a polymer comprised of silicone monomers and monomers containing amide groups as further described herein. The silicone polyamide preferably comprises moieties of the general formula:

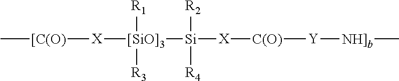

X is a linear or branched alkylene having from about 1-30 carbon atoms; R₁, R₂, R₃, and R₄ are each independently C₁₋₃₀ straight or branched chain alkyl which may be substituted with one or more hydroxyl or halogen groups; phenyl which may be substituted with one or more C₁₋₃₀ alkyl groups, halogen, hydroxyl, or alkoxy groups; or a siloxane chain having the general formula:

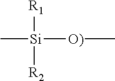

### and Y is:

(a) a linear or branched alkylene having from about 1-40 carbon atoms which may be substituted with:
   (i) one or more amide groups having the general formula R₁CONR₁, or
   (ii) C₅₋₆ cyclic ring, or
   (iii) phenylene which may be substituted with one or more C₁₋₁₀ alkyl groups, or
   (iv) hydroxy, or
   (v) C₃₋₈ cycloalkane, or
   (vi) C₁₋₂₀ alkyl which may be substituted with one or more hydroxy groups, or
   (vii) C₁₋₁₀ alkyl amines; or
(b) TR₅R₆R₇
   wherein R₅, R₆, and R₇, are each independently a C₁₋₁₀ linear or branched alkylenes, and T is CR₈ wherein R₈ is hydrogen, a trivalent atom N, P, or Al, or a C₁₋₃₀ straight or branched chain alkyl which may be substituted with one or more hydroxyl or halogen groups; phenyl which may be substituted with one or more C₁₋₃₀ alkyl groups, halogen, hydroxyl, or alkoxy groups; or a siloxane chain having the general formula:

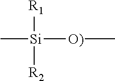

Preferred is where R₁, R₂, R₃, and R₄ are C₁₋₁₀, preferably methyl; and X and Y is a linear or branched alkylene. Preferred are silicone polyamides having the general formula:

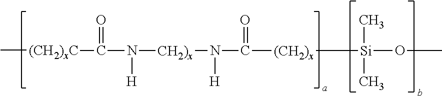

wherein a and b are each independently sufficient to provide a silicone polyamide polymer having a melting point ranging from about 60 to 120° C., and a molecular weight ranging from about 40,000 to 500,000 Daltons. One type of silicone polyamide that may be used in the compositions of the invention may be purchased from Dow Corning Corporation under the tradename Dow Corning 2-8178 gellant which has the CTFA name nylon-611/dimethicone copolymer which is sold in a composition containing PPG-3 myristyl ether.

Also suitable are polyamides such as those purchased from Arizona Chemical under the tradenames Uniclear and Sylvaclear. Such polyamides may be ester terminated or amide terminated. Examples of ester terminated polyamides include, but are not limited to those having the general formula:

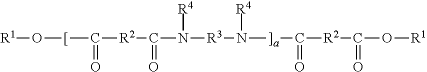

wherein n denotes a number of amide units such that the number of ester groups ranges from about 10% to 50% of the total number of ester and amide groups; each R₁ is independently an alkyl or alkenyl group containing at least 4 carbon atoms; each R₂ is independently a C₄₋₄₂ hydrocarbon group, with the proviso that at least 50% of the R₂ groups are a C30-42 hydrocarbon; each R₃ is independently an organic group containing at least 2 carbon atoms, hydrogen atoms and optionally one or more oxygen or nitrogen atoms; and each R₄ is independently a hydrogen atom, a C₁₋₁₀ alkyl group or a direct bond to R₃ or to another R₄, such that the nitrogen atom to which R₃ and R₄ are both attached forms part of a heterocyclic structure defined by R₄—N—R₃, with at least 50% of the groups R₄ representing a hydrogen atom.

General examples of ester and amide terminated polyamides that may be used as oil phase gelling agents include those sold by Arizona Chemical under the tradenames Sylvaclear A200V or A2614V, both having the CTFA name ethylenediamine/hydrogenated dimer dilinoleate copolymer/bis-di-C₁₄₋₁₈ alkyl amide; Sylvaclear AF1900V; Sylvaclear C75V having the CTFA name bis-stearyl ethylenediamine/neopentyl glycol/stearyl hydrogenated dimer dilinoleate copolymer; Sylvaclear PA1200V having the CTFA name Polyamide-3; Sylvaclear PE400V; Sylvaclear WF1500V; or Uniclear, such as Uniclear 100VG having the INCI name ethylenediamine/stearyl dimer dilinoleate copolymer; or ethylenediamine/stearyl dimer ditallate copolymer. Other examples of suitable polyamides include those sold by Henkel under the Versamid trademark (such as Versamid 930, 744, 1655), or by Olin Mathieson Chemical Corp. under the brand name Onamid S or Onamid C.

(iv). Natural or Synthetic Organic Waxes

Also suitable as the oil phase structuring agent may be one or more natural or synthetic waxes such as animal, vegetable, or mineral waxes. Preferably such waxes will have a higher melting point such as from about 50 to 150° C., more preferably from about 65 to 100° C. Examples of such waxes include waxes made by Fischer-Tropsch synthesis, such as polyethylene or synthetic wax; or various vegetable waxes such as bayberry, candelilla, ozokerite, acacia, beeswax, ceresin, cetyl esters, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, grape wax, and polyalkylene glycol derivatives thereof such as PEG 6-20 beeswax, or PEG-12 carnauba wax; or fatty acids or fatty alcohols, including esters thereof, such as hydroxystearic acids (for example 12-hydroxy stearic acid), tristearin, tribehenin, and so on.

(v). Montmorillonite Minerals

One type of structuring agent that may be used in the composition comprises natural or synthetic montmorillonite minerals such as hectorite, bentonite, and quaternized derivatives thereof, which are obtained by reacting the minerals with a quaternary ammonium compound, such as stearalkonium bentonite, hectorites, quaternized hectorites such as Quaternium-18 hectorite, attapulgite, carbonates such as propylene carbonate, bentones, and the like.

(vi). Silicas and Silicates

Another type of structuring agent that may be used in the compositions are silicas, silicates, silica silylate, and alkali metal or alkaline earth metal derivatives thereof. These silicas and silicates are generally found in the particulate form and include silica, silica silylate, magnesium aluminum silicate, and the like.

5. Surfactants

The composition may contain one or more surfactants, especially if in the emulsion form. However, such surfactants may be used if the compositions are anhydrous also, and will assist in dispersing ingredients that have polarity, for example pigments. Such surfactants may be silicone or organic based. The surfactants will aid in the formation of stable emulsions of either the water-in-oil or oil-in-water form. If present, the surfactant may range from about 0.001 to 30%, preferably from about 0.005 to 25%, more preferably from about 0.1 to 20% by weight of the total composition.

(a). Silicone Surfactants

Suitable silicone surfactants include polyorganosiloxane polymers that have amphiphilic properties, for example contain hydrophilic radicals and lipophilic radicals. These silicone surfactants may be liquids or solids at room temperature.

(i). Dimethicone Copolyols or Alkyl Dimethicone Copolyols

One type of silicone surfactant that may be used is generally referred to as dimethicone copolyol or alkyl dimethicone copolyol. This surfactant is either a water-in-oil or oil-in-water surfactant having an Hydrophile/Lipophile Balance (HLB) ranging from about 2 to 18. Preferably the silicone surfactant is a nonionic surfactant having an HLB ranging from about 2 to 12, preferably about 2 to 10, most preferably about 4 to 6. The term “hydrophilic radical” means a radical that, when substituted onto the organosiloxane polymer backbone, confers hydrophilic properties to the substituted portion of the polymer. Examples of radicals that will confer hydrophilicity are hydroxy-polyethyleneoxy, hydroxyl, carboxylates, and mixtures thereof. The term “lipophilic radical” means an organic radical that, when substituted onto the organosiloxane polymer backbone, confers lipophilic properties to the substituted portion of the polymer. Examples of organic radicals that will confer lipophilicity are C₁₋₄₀ straight or branched chain alkyl, fluoro, aryl, aryloxy, C₁₋₄₀ hydrocarbyl acyl, hydroxy-polypropyleneoxy, or mixtures thereof.

One type of suitable silicone surfactant has the general formula:

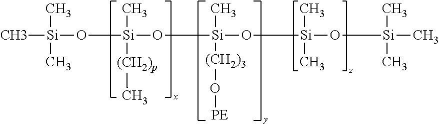

wherein p is 0-40 (the range including all numbers between and subranges such as 2, 3, 4, 13, 14, 15, 16, 17, 18, etc.), and PE is (—C₂H₄O)ₐ—(—C₃H₆O)_{b}—H wherein a is 0 to 25, b is 0-25 with the proviso that both a and b cannot be 0 simultaneously, x and y are each independently ranging from 0 to 1 million with the proviso that they both cannot be 0 simultaneously. In one preferred embodiment, x, y, z, a, and b are such that the molecular weight of the polymer ranges from about 5,000 to about 500,000, more preferably from about 10,000 to 100,000, and is most preferably approximately about 50,000 and the polymer is generically referred to as dimethicone copolyol.

One type of silicone surfactant is wherein p is such that the long chain alkyl is cetyl or lauryl, and the surfactant is called, generically, cetyl dimethicone copolyol or lauryl dimethicone copolyol respectively.

In some cases the number of repeating ethylene oxide or propylene oxide units in the polymer are also specified, such as a dimethicone copolyol that is also referred to as PEG-15/PPG-10 dimethicone, which refers to a dimethicone having substituents containing 15 ethylene glycol units and 10 propylene glycol units on the siloxane backbone. It is also possible for one or more of the methyl groups in the above general structure to be substituted with a longer chain alkyl (e.g. ethyl, propyl, butyl, etc.) or an ether such as methyl ether, ethyl ether, propyl ether, butyl ether, and the like.

Examples of silicone surfactants are those sold by Dow Corning under the tradename Dow Corning 3225C Formulation Aid having the CTFA name cyclotetrasiloxane (and) cyclopentasiloxane (and) PEG/PPG-18 dimethicone; or 5225C Formulation Aid, having the CTFA name cyclopentasiloxane (and) PEG/PPG-18/18 dimethicone; or Dow Corning 190 Surfactant having the CTFA name PEG/PPG-18/18 dimethicone; or Dow Corning 193 Fluid, Dow Corning 5200 having the CTFA name lauryl PEG/PPG-18/18 methicone; or Abil EM 90 having the CTFA name cetyl PEG/PPG-14/14 dimethicone sold by Goldschmidt; or Abil EM 97 having the CTFA name bis-cetyl PEG/PPG-14/14 dimethicone sold by Goldschmidt; or Abil WE 09 having the CTFA name cetyl PEG/PPG-10/1 dimethicone in a mixture also containing polyglyceryl-4 isostearate and hexyl laurate; or KF-6011 sold by Shin-Etsu Silicones having the CTFA name PEG-11 methyl ether dimethicone; KF-6012 sold by Shin-Etsu Silicones having the CTFA name PEG/PPG-20/22 butyl ether dimethicone; or KF-6013 sold by Shin-Etsu Silicones having the CTFA name PEG-9 dimethicone; or KF-6015 sold by Shin-Etsu Silicones having the CTFA name PEG-3 dimethicone; or KF-6016 sold by Shin-Etsu Silicones having the CTFA name PEG-9 methyl ether dimethicone; or KF-6017 sold by Shin-Etsu Silicones having the CTFA name PEG-10 dimethicone; or KF-6038 sold by Shin-Etsu Silicones having the CTFA name lauryl PEG-9 polydimethylsiloxyethyl dimethicone.

(ii). Crosslinked Silicone Surfactants

Also suitable are various types of crosslinked silicone surfactants that are often referred to as emulsifying elastomers. They are typically prepared as set forth above with respect to the section “silicone elastomers” except that the silicone elastomers will contain at least one hydrophilic moiety such as polyoxyalkylenated groups. Typically these polyoxyalkylenated silicone elastomers are crosslinked organopolysiloxanes that may be obtained by a crosslinking addition reaction of diorganopolysiloxane comprising at least one hydrogen bonded to silicon and of a polyoxyalkylene comprising at least two ethylenically unsaturated groups. In at least one embodiment, the polyoxyalkylenated crosslinked organopolysiloxanes are obtained by a crosslinking addition reaction of a diorganopolysiloxane comprising at least two hydrogens each bonded to a silicon, and a polyoxyalkylene comprising at least two ethylenically unsaturated groups, optionally in the presence of a platinum catalyst, as described, for example, in U.S. Pat. No. 5,236,986 and U.S. Pat. No. 5,412,004, U.S. Pat. No. 5,837,793 and U.S. Pat. No. 5,811,487, the contents of which are incorporated by reference.

Polyoxyalkylenated silicone elastomers that may be used in at least one embodiment of the invention include those sold by Shin-Etsu Silicones under the names KSG-21, KSG-20, KSG-30, KSG-31, KSG-32, KSG-33; KSG-210 which is dimethicone/PEG-10/15 crosspolymer dispersed in dimethicone; KSG-310 which is PEG-15 lauryl dimethicone crosspolymer; KSG-320 which is PEG-15 lauryl dimethicone crosspolymer dispersed in isododecane; KSG-330 (the former dispersed in triethylhexanoin), KSG-340 which is a mixture of PEG-10 lauryl dimethicone crosspolymer and PEG-15 lauryl dimethicone crosspolymer.

Also suitable are polyglycerolated silicone elastomers like those disclosed in PCT/WO 2004/024798, which is hereby incorporated by reference in its entirety. Such elastomers include Shin-Etsu's KSG series, such as KSG-710 which is dimethicone/polyglycerin-3 crosspolymer dispersed in dimethicone; or lauryl dimethicone/polyglycerin-3 crosspolymer dispersed in a variety of solvent such as isododecane, dimethicone, triethylhexanoin, sold under the Shin-Etsu tradenames KSG-810, KSG-820, KSG-830, or KSG-840. Also suitable are silicones sold by Dow Corning under the tradenames 9010 and DC9011.

One preferred crosslinked silicone elastomer emulsifier is dimethicone/PEG-10/15 crosspolymer, which provides excellent aesthetics due to its elastomeric backbone, but also surfactancy properties.

(iii). Organic Nonionic Surfactants

The composition may comprise one or more nonionic organic surfactants. Suitable nonionic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Steareth 2-100, which is formed by the reaction of stearyl alcohol and ethylene oxide and the number of ethylene oxide units ranges from 2 to 100; Beheneth 5-30 which is formed by the reaction of behenyl alcohol and ethylene oxide where the number of repeating ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on.

Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol. Examples include polymeric alkylene glycols reacted with glyceryl fatty acid esters such as PEG glyceryl oleates, PEG glyceryl stearate; or PEG polyhydroxyalkanotes such as PEG dipolyhydroxystearate wherein the number of repeating ethylene glycol units ranges from 3 to 1000.

Also suitable as nonionic surfactants are formed by the reaction of a carboxylic acid with an alkylene oxide or with a polymeric ether. The resulting products have the general formula:

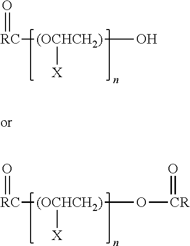

where RCO is the carboxylic ester radical, X is hydrogen or lower alkyl, and n is the number of polymerized alkoxy groups. In the case of the diesters, the two RCO-groups do not need to be identical. Preferably, R is a C6-30 straight or branched chain, saturated or unsaturated alkyl, and n is from 1-100.

Monomeric, homopolymeric, or block copolymeric ethers are also suitable as nonionic surfactants. Typically, such ethers are formed by the polymerization of monomeric alkylene oxides, generally ethylene or propylene oxide. Such polymeric ethers have the following general formula:

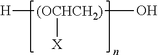

wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular ethoxylation of sorbitan provides polyalkoxylated sorbitan derivatives. Esterification ofpolyalkoxylated sorbitan provides sorbitan esters such as the polysorbates. For example, the polyalkyoxylated sorbitan can be esterified with C6-30, preferably C12-22 fatty acids. Examples of such ingredients include Polysorbates 20-85, sorbitan oleate, sorbitan sesquioleate, sorbitan palmitate, sorbitan sesquiisostearate, sorbitan stearate, and so on.

Certain types of amphoteric, zwitterionic, or cationic surfactants may also be used in the compositions. Descriptions of such surfactants are set forth in U.S. Pat. No. 5,843,193, which is hereby incorporated by reference in its entirety.

6. Sunscreens

It may also be desirable to include one or more sunscreens in the compositions of the invention. Such sunscreens include chemical UVA or UVB sunscreens or physical sunscreens in the particulate form.

(a). UVA Chemical Sunscreens

If desired, the composition may comprise one or more UVA sunscreens. The term “UVA sunscreen” means a chemical compound that blocks UV radiation in the wavelength range of about 320 to 400 nm. Preferred UVA sunscreens are dibenzoylmethane compounds having the general formula:

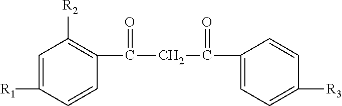

wherein R₁ is H, OR and NRR wherein each R is independently H, C₁₋₂₀ straight or branched chain alkyl; R₂ is H or OH; and R₃ is H, C₁₋₂₀ straight or branched chain alkyl.

Preferred is where R₁ is OR where R is a C₁₋₂₀ straight or branched alkyl, preferably methyl; R₂ is H; and R₃ is a C₁₋₂₀ straight or branched chain alkyl, more preferably, butyl.

Examples of suitable UVA sunscreen compounds of this general formula include 4-methyldibenzoylmethane, 2-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′diisopropylbenzoylmethane, 4-tert-butyl-4′-methoxydibenzoylmethane, 4,4′-diisopropylbenzoylmethane, 2-methyl-5-isopropyl-4′-methoxydibenzoymethane, 2-methyl-5-tert-butyl-4′-methoxydibenzoylmethane, and so on. Particularly preferred is 4-tert-butyl-4′-methoxydibenzoylmethane, also referred to as Avobenzone. Avobenzone is commercial available from Givaudan-Roure under the trademark Parsol 1789, and Merck & Co. under the tradename Eusolex 9020.

Other types of UVA sunscreens include dicamphor sulfonic acid derivatives, such as ecamsule, a sunscreen sold under the trade name Mexoryl™, which is terephthalylidene dicamphor sulfonic acid, having the formula:

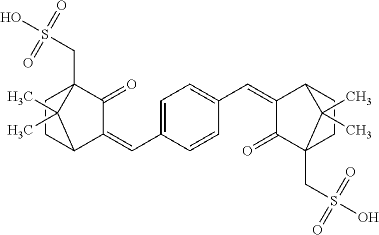 [SMILES: CC1(C)C2CCC1(CS(=O)(=O)O)C(=O)/C2=C\c1ccc(/C=C2\C(=O)C3(CS(=O)(=O)O)CCC2C3(C)C)cc1]

The composition may contain from about 0.001-20%, preferably 0.005-5%, more preferably about 0.005-3% by weight of the composition of UVA sunscreen. In the preferred embodiment of the invention the UVA sunscreen is Avobenzone, and it is present at not greater than about 3% by weight of the total composition.

(b). UVB Chemical Sunscreens

The term “UVB sunscreen” means a compound that blocks UV radiation in the wavelength range of from about 290 to 320 nm. A variety of UVB chemical sunscreens exist including alpha-cyano-beta,beta-diphenyl acrylic acid esters as set forth in U.S. Pat. No. 3,215,724, which is hereby incorporated by reference in its entirety. One particular example of an alpha-cyano-beta,beta-diphenyl acrylic acid ester is Octocrylene, which is 2-ethylhexyl 2-cyano-3,3-diphenylacrylate. In certain cases the composition may contain no more than about 110% by weight of the total composition of octocrylene. Suitable amounts range from about 0.001-10% by weight. Octocrylene may be purchased from BASF under the tradename Uvinul N-539.

Other suitable sunscreens include benzylidene camphor derivatives as set forth in U.S. Pat. No. 3,781,417, which is hereby incorporated by reference in its entirety. Such benzylidene camphor derivatives have the general formula:

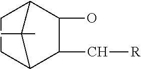

wherein R is p-tolyl or styryl, preferably styryl. Particularly preferred is 4-methylbenzylidene camphor, which is a lipid soluble UVB sunscreen compound sold under the tradename Eusolex 6300 by Merck.

Also suitable are cinnamate derivatives having the general formula:

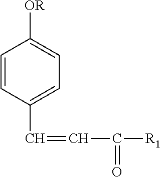

wherein R and R₁ are each independently a C₁₋₂₀ straight or branched chain alkyl. Preferred is where R is methyl and R₁ is a branched chain C₁₋₁₀, preferably C₈ alkyl. The preferred compound is ethylhexyl methoxycinnamate, also referred to as Octoxinate or octyl methoxycinnamate. The compound may be purchased from Givaudan Corporation under the tradename Parsol MCX, or BASF under the tradename Uvinul MC80. Also suitable are mono-, di-, and triethanolamine derivatives of such methoxy cinnamates including diethanolamine methoxycinnamate. Cinoxate, the aromatic ether derivative of the above compound is also acceptable. If present, the Cinoxate should be found at no more than about 3% by weight of the total composition.

Also suitable as UVB screening agents are various benzophenone derivatives having the general formula:

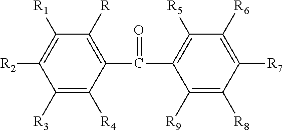

wherein R through R₉ are each independently H, OH, NaO₃S, SO₃H, SO₃Na, Cl, R″, OR″ where R″ is C₁₋₂₀ straight or branched chain alkyl Examples of such compounds include Benzophenone 1, 2, 3, 4, 5, 6, 7, 8, 9, 10, 11, and 12. Particularly preferred is where the benzophenone derivative is Benzophenone 3 (also referred to as Oxybenzone), Benzophenone 4 (also referred to as Sulisobenzone), Benzophenone 5 (Sulisobenzone Sodium), and the like. Most preferred is Benzophenone 3.

Also suitable are certain menthyl salicylate derivatives having the general formula:

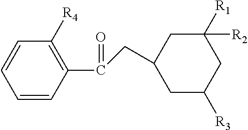

wherein R₁, R₂, R₃, and R₄ are each independently H, OH, NH₂, or C₁₋₂₀ straight or branched chain alkyl. Particularly preferred is where R₁, R₂, and R₃ are methyl and R₄ is hydroxyl or NH₂, the compound having the name homomenthyl salicylate (also known as Homosalate) or menthyl anthranilate. Homosalate is available commercially from Merck under the tradename Eusolex HMS and menthyl anthranilate is commercially available from Haarmann & Reimer under the tradename Heliopan. If present, the Homosalate should be found at no more than about 15% by weight of the total composition.

Various amino benzoic acid derivatives are suitable UVB absorbers including those having the general formula:

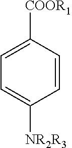

wherein R₁, R₂, and R₃ are each independently H, C₁₋₂₀ straight or branched chain alkyl which may be substituted with one or more hydroxy groups. Particularly preferred is wherein R₁ is H or C₁₋₈ straight or branched alkyl, and R₂ and R₃ are H, or C₁₋₈ straight or branched chain alkyl. Particularly preferred are PABA, ethyl hexyl dimethyl PABA (Padimate O), ethyldihydroxypropyl PABA, and the like. If present Padimate O should be found at no more than about 8% by weight of the total composition.

Salicylate derivatives are also acceptable UVB absorbers. Such compounds have the general formula:

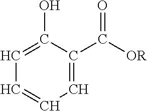

wherein R is a straight or branched chain alkyl, including derivatives of the above compound formed from mono-, di-, or triethanolamines. Particular preferred are octyl salicylate, TEA-salicylate, DEA-salicylate, and mixtures thereof.
Generally, the amount of the UVB chemical sunscreen present may range from about 0.001-45%, preferably 0.005-40%, more preferably about 0.01-35% by weight of the total composition.

Also suitable are particulate sunscreens such as zinc oxide or titanium dioxide, which may have particle sizes ranging from 0.1 to 100 microns.

If desired, the compositions of the invention may be formulated to have a certain SPF (sun protective factor) values ranging from about 1-50, preferably about 2-45, most preferably about 5-30. Calculation of SPF values is well known in the art.

7. Particulate Materials

The compositions of the invention may contain particulate materials in the form of pigments, inert particulates, or mixtures thereof. If present, suggested ranges are from about 0.01-75%, preferably about 0.5-70%, more preferably about 0.1-65% by weight of the total composition. In the case where the composition may comprise mixtures of pigments and powders, suitable ranges include about 0.01-75% pigment and 0.1-75% powder, such weights by weight of the total composition.

(a). Powders

The particulate matter may be colored or non-colored (for example white) non-pigmented powders. Suitable non-pigmented powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. The above mentioned powders may be surface treated with lecithin, amino acids, mineral oil, silicone, or various other agents either alone or in combination, which coat the powder surface and render the particles more lipophilic in nature.

(b). Pigments

The particulate materials may comprise various organic and/or inorganic pigments. The organic pigments are generally various aromatic types including azo, indigoid, triphenylmethane, anthroquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof. Iron oxides of red, blue, yellow, brown, black, and mixtures thereof are suitable.

8. Preservatives

The composition may contain 0.001-8%, preferably 0.01-6%, more preferably 0.05-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, caprylyl glycol, biguanide derivatives, phenoxyethanol, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and the like. In one preferred embodiment the composition is free of parabens.

9. Vitamins and Antioxidants

The compositions of the invention may contain vitamins and/or coenzymes, as well as antioxidants. If so, 0.001-10%, preferably 0.01-8%, more preferably 0.05-5% by weight of the total composition is suggested. Suitable vitamins include ascorbic acid and derivatives thereof such as ascorbyl palmitate, tetrahexydecyl ascorbate, and so on; the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are retinyl palmitate, retinol. retinoic acid, as well as Vitamin A in the form of beta carotene. Also suitable is Vitamin E and derivatives thereof such as Vitamin E acetate, nicotinate, or other esters thereof. In addition, Vitamins D and K are suitable.

Suitable antioxidants are ingredients which assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

### II. The Form of the Compositions

The compositions may be in the form of anhydrous compositions, solutions, or emulsions in water-in-oil or oil-in-water form. The compositions may be liquid, solid, or semi-solid. If in the form of emulsions, from about 0.1 to 80% water and from about 0.1 to 80% water may be present. If in the anhydrous form the compositions typically contain oil and other ingredients such as powders, pigments, oils, or structuring agents. If in the solution form the compositions contain from about 0.1 to 80% water and other water soluble or dispersible ingredients.

The compositions may be in the form of those applied to skin such as creams, lotions, serums, gels, etc., or color cosmetics such as foundations, concealers, eye shadow, eye liner, mascara, blush, lipstick, lip gloss, and the like.

In one preferred embodiment of the invention the volume increase agent or volume decrease agent may be in the form of a skin cream comprising:

10-85% water,

0.5-40% oil; and

1-25% of the volume increase agent or the volume decrease agent.

In another preferred embodiment the volume increase agent or the volume decrease agent may be in the form of a primer in oil in water emulsion form for application to skin or under eye area comprising:

10-80% water,

1-35% oil,

1-20% volume increase or volume decrease agent; and

1-35% particulates comprising pigments and powders.

In another preferred embodiment the composition is a primer that changes color upon application to skin from a first resting color to a second application color, comprising:

10-85% water,

1-20% oil; and

0.5-20% of the volume increase or volume decrease agent.

In another preferred embodiment the composition is a serum comprising:

5-90% water,

3-25% glycerin,

0.5-20% oil; and

0.5-20% of the volume increase or volume decrease ingredient.

In another preferred embodiment the composition is a cream or lotion in oil in water emulsion form comprising:

1-85% water,

0.1-25% oil; and

0.1-20% of the volume increase or volume decrease agent.

In addition to the above, the compositions of the invention can exist in a variety of other forms, such as emulsions, suspensions, dispersions, solutions, and anhydrous compositions.

The invention will be further described in connection with the following examples which are set forth for the purposes of illustration only.

### Example 1

Compositions for use in the method and kit of the invention are set forth below:

| | Volume | Volume | Volume |
|---|---|---|---|
| | Increase | Decrease | Increase |
| Ingredient | Formula | Formula | Formula |
| Water | QS | QS | QS |
| Triethylhexanoin | | 8.00 | |
| Jojoba oil | | 4.79 | |
| Coco-caprylate/caprate | 5.90 | | |
| C12-20 acid PEG-8 ester | 4.96 | | |
| Glycerin | 2.00 | 4.00 | |
| Squalane | | 4.00 | |
| Batyl alcohol/stearic acid/caprylic/capric | | 2.50 | |
| triglyceride/lecithin | | | |
| Tocopherol acetate | | 0.50 | 0.50 |
| Water/ergothioneine | | 1.00 | 1.00 |
| Cetyl alcohol | 2.00 | | |
| Cocos nucifera (coconut) oil | 1.60 | | |
| Butylene glycol | 1.00 | | 1.10 |
| Glycereth-26 | | | 1.00 |
| Ethylhexylglycerin | 1.00 | | |
| PEG-100 stearate | 0.75 | 0.30 | |
| PEG-75 | | | 4.00 |
| Squalane | | | 0.50 |
| Methyl gluceth-20 | | | 4.00 |
| Bis-PEG-18 methyl ether dimethyl silane | | | 2.00 |
| Phytantriol | 0.50 | | |
| Sodium dehydroacetate | | 0.10 | 0.10 |
| Sodium PCA | 0.50 | | |
| Acetyl glucosamine | 0.50 | | |
| Whey protein | 0.50 | | |
| Milk protein | 0.50 | | |
| Glucosamine HCL | 0.50 | | |
| Trehalose | 0.50 | 0.10 | |
| Ammonium acryloyldimethyltaurate/VP | 0.50 | | |
| Copolymer | | | |
| Dimethicone | 0.50 | 0.40 | |
| Pentylene glycol | 0.50 | 1.50 | |
| Phenoxyethanol | 0.32 | 0.20 | 0.60 |
| Triethanolamine | | | 0.50 |
| Pantethine | | | 0.14 |
| Caffeine | 0.20 | 0.20 | 0.20 |
| Oleth-3 phosphate | | | 0.45 |
| Carbomer | | | 0.40 |
| Oleth-3 | | | 0.35 |
| Oleth-5 | | | 0.24 |
| Caprylic/capric triglyceride/Commiphora | | | 0.50 |
| mukul resin | | | |
| Siegesbeckia orientalis extract/glycerin | 0.20 | | |
| Wheat bran extract/olive extract | 0.20 | | |
| Potassium cetyl phosphate | 0.20 | | |
| Anogeissus leiocarpus bark extract | 0.15 | | |
| Artemia extract | | | 1.00 |
| Citrullus vulgaris/Pyrus malus fruit | | | 1.00 |
| extract/Lens esculenta fruit extract/sodium | | | |
| lactate/sodium PCA | | | |
| Betula alba extract | 0.10 | | |
| Tamarindus indica seed extract | 0.10 | | |
| Argania spinosa leaf extract | 0.10 | | |
| Larix sibrica wood extract | 0.10 | | |
| Caprylic/capric triglyceride/Dictyopteris | | | 0.50 |
| membranacea extract | | | |
| Centaurium erythraea extract | 0.10 | | |
| Laminaria saccharina | 0.10 | | |
| Laminaria digitata extract | | 0.50 | |
| Coleus barbatus extract | | 0.20 | |
| Hydrogenated lecithin | | 0.50 | |
| Dipotassium glycyrrhizate | | | 0.10 |
| Aminopropyl ascorbyl phosphate | 0.10 | | |
| Propylene glycol dicaprylate/cucumber | | 0.10 | |
| fruit extract/avocado | | | |
| oil/cholesterol/potassium sulfate | | | |
| Propylene glycol dicaprate/Helianthus | 0.10 | | |
| annus seed cake/Barley extract/cucumber | | | |
| fruit extract | | | |
| Xanthan gum | 0.08 | 0.20 | |
| Magnolia grandiflora bark extract | | 0.05 | |
| Isomerized linoleic acid/linoleic acid | | 0.03 | |
| Citric acid | | 0.02 | 0.02 |
| Sodium dehydroacetate | 0.05 | | |
| Sodium hyaluronate | | | 0.02 |
| Cholesterol/potassium sulfate | 0.05 | | |
| Sodium hyaluronate | 0.02 | 0.10 | |
| Garcinia indica seed butter | | 0.01 | |
| Ethylhexyl glycerin | | | 0.50 |
| Bisabolol | | | 0.20 |
| Resveratrol ferulate/caprylic capric | | 0.01 | |
| triglyceride | | | |
| Trisodium EDTA | | | 0.10 |
| Choleth-24/Ceteth-24 | | | 0.10 |
| Acrylates C10-20 alkyl acrylate | | 0.02 | |
| crosspolymer | | | |
| Sodium acrylate/sodium acryloyldimethyl | 0.001 | | |
| taurate copolymer/hydrogenated | | | |
| polydecene/laureth-8 | | | |

The compositions were prepared by separately mixing the water and oil phase ingredients then emulsifying to form an emulsion. The compositions were stored in containers.

### Example 2

Ingredients having adipogenesis (volume increasing) activity were tested as follows:

### Compositions Tested:

(a) Commipheroline (90 parts caprylic/capric triglyceride & 10 parts Commiphora mukul resin extract);
(b) Rosiglatizone, potassium salt. 5-[[4-[2-methyl-2-pyridinylamino)ethoxyphenyl]methyl]-2,4-thizaolidinedione, potassium salt;

### (c) Dimethylsulfoxide (DMSO).

Cell culture, treatment and differentiation was performed using kit purchased from Zenbio, Subcutaneous Human Adipocytes (Maintenance and Differentiation from Preadipocytes to Adipocytes). The donor adipocytes used were from 41 year old female with a BMI of 25.2; lot number L061103, cultured in 96 well plates.

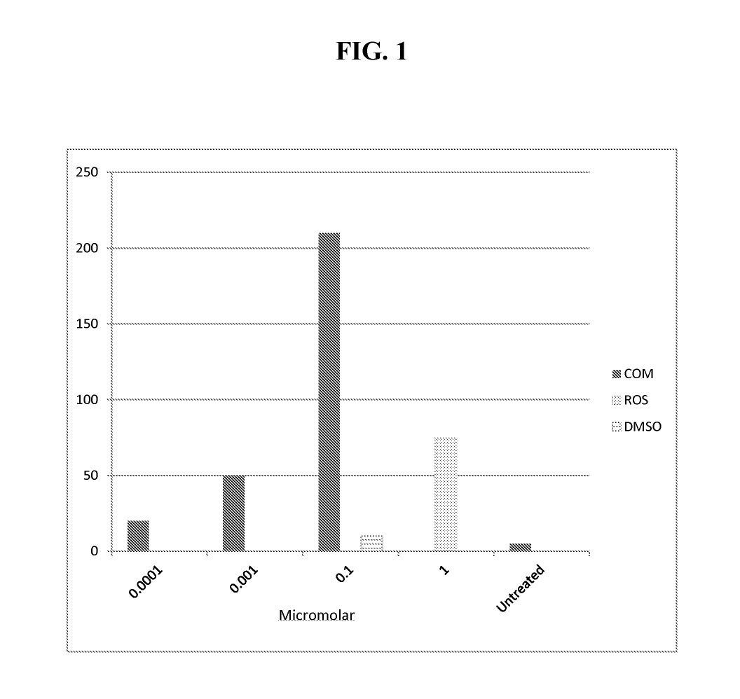

The results are set forth in FIG. 1 and show that Commipheroline (“COM”) is effective in increasing lipid production in human adipocytes, which is an indicator of increasing the appearance of volume on the skin surface to which the resulting composition is applied when compared with Rosiglitazone (ROS) and control (DMSO=dimethyl sulfoxide).

### Example 3

Conjugated linoleic acid and resveratrol were tested for their ability to promote the appearance of decreased volume. Cell culture, treatment, and differentiation assay was performed by Zen-Bio, RTF, NC. Human subcutaneous preadipocytes, lot number: were cultured in 96-well plates. The donor information is as follows:
Lot #: L061103
Sex: Female
Age: 41
B.M.I.: 25.2
   Stimulation was treated in the presence of 0.1 uM Rosiglitazone Maleate.
   Inhibition was treated in the presence of 1.0 uM Rosiglitazone Maleate.
   Day 1: Plated cells and allowed cells to adhere.
   Day 2: Changed medium to Initiation medium containing the compounds or controls.
   Day 5: Changed medium to Adipocyte medium with the compounds.
   Day 8: Changed medium to Adipocyte medium with the compounds.
   Day 11: Changed medium to Adipocyte medium with the compounds.
   Day 13: Cells were washed with PBS and lysed with the lysis buffer. Reagent B was added to each well and the cells were incubated at 37° C. for 2 hours. Aliquots of the lysates were diluted with PBS, then Reagent A was added to each well. The optical density was read at 540 nM after 15 minutes. The reagent measures the amount of glycerol liberated from triglyceride.

### Statistical Analysis

Data were analyzed using a one-way ANOVA and significant means were tested by Bonferroni multiple comparison test using Instat software.

Cell culture, treatment, and differentiation assay was performed by Zen-Bio, RTF, NC. Human subcutaneous preadipocytes, lot number: were cultured in 96-well plates. The donor information is as follows:
Lot#: SL0029
Gender: Female (all lots)
Age: 44 (avg)
B.M.I.: 26.98 (avg)

### Cell Culture and Treatment

Human subcutaneous preadipocytes were cultured in 96-well plates.
Day 1: Plated cells and allowed cells to adhere.
Day 2: Changed medium to Differentiation medium containing the compounds or controls. (0.1 uM Rosi for stimulation or 1 uM Rosi for inhibition)
Day 9: Changed medium to Adipocyte medium plus compounds.
Day 12: Changed medium to Adipocyte medium plus compounds.
Day 16: Cells were washed with PBS and lysed with the lysis buffer. Reagent B was added to each well and the cells were incubated at 37° C. for 2 hours. Aliquots of the lysates were diluted with PBS, then Reagent A was added to each well. The optical density was read at 540 nM after 15 minutes. The reagent measures the amount of glycerol liberated from triglyceride.

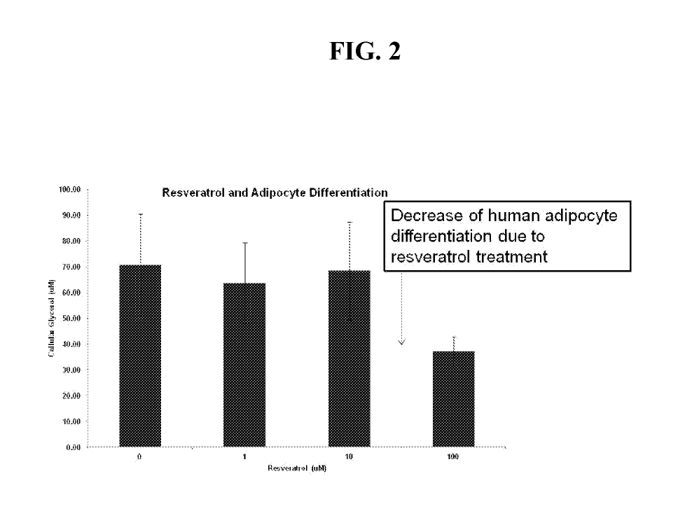

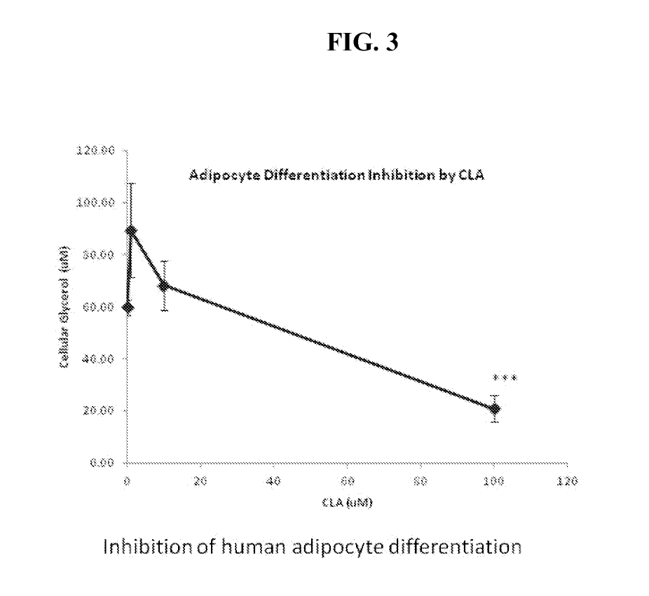

The results are set forth in FIGS. 2 and 3 and show that both resveratrol and conjugated linoleic acid are effective in reducing adipogenesis, which is correlated with a reduction in appearance of volume on the skin surface to which it is applied.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

What we claim is:

1. A method for modulating the appearance of volume on select facial or body surfaces in need thereof comprising:
(a) identifying the surface for which modulating by increasing the appearance of volume is desired;
(b) if such surface is identified, treating the surface of (a) with an agent that causes the appearance of increased volume to the area of treatment;
(c) identifying the surfaces for which modulating by decreasing the appearance of volume is desired;
(d) if such surface is identified, treating the surface of (c) with an agent that causes the appearance of decreased volume to the area of treatment.

2. The method of claim 1 wherein the select facial or body surfaces are treated to increase the appearance of volume.

3. The method of claim 1 wherein the select facial or body surfaces are treated to decrease the appearance of volume.

4. The method of claim 1 wherein some select facial or body surfaces are treated to increase the appearance of volume, and other select facial or body surfaces are treated to decrease the appearance of volume.

5. The method of claim 1 wherein the agent that increases the appearance of volume is a topically applied composition.

6. The method of claim 5 wherein the topically applied composition stimulates adipogenesis.

7. The method of claim 6 wherein the adipogenesis stimulating composition comprises one or adipogenesis stimulators selected from Commipheroline (Commiphora mukul extract); extract of Anemarrhena asphodeloides Sarsasapogenin Adansonia digitata pulp extract; Hibiscus sabdariffa flower extract; Macadamia Ternifolia Seed Oil; Sargaquinoic acid (SQA); sargahydroquinoic acid (SHQA); Sargassum yezoense extract; C6-12 fatty carboxylic acids; acetyl hexapeptide-38; Inonotus Obliquus (Chaga mushroom) extract: Bixa orellana extract (from Achiote plant): beta glucan and mixtures thereof.

8. The method of claim 7 wherein the adipogenesis stimulating composition comprises Commiphora mukul extract.

9. The method of claim 1 wherein the volume increase agent is physical manipulation.

10. The method of claim 9 wherein the physical manipulation is massage or electronic muscle stimulation.

11. The method of claim 1 wherein the volume decrease agent is a topically applied composition.

12. The method of claim 11 wherein the volume decrease composition comprises a volume decrease ingredient selected from the group of an adipogenesis inhibitor, an adipocyte lipolytic agent, or a skin tightening agent.

13. The method of claim 12 wherein the volume decrease agent is a composition.

14. The method of claim 12 wherein the volume decrease agent is an ingredient is selected from conjugated linoleic acid; resveratrol; esters of resveratrol and inorganic acids; Myrtus Communis leaf extract; extracts of plants from the Acacia genus; Calluna Vulgaris; Rosa Canina fruit extract; nettle leaf extract; Polyporus Umbellatus; Chamomilla Recutita (Matricaria) flower oil; Coleus Forskohlii extract; a mixture of Butcher's Broom extract and hesperidin methyl chalcone; HOP's beta acids; tripeptides of the sequence Val-Tyr-Pro or Val-Thr-Leu; Haplophyllum hispanicum Spach; Scutellaria Rivularis extract; alpha ketoboswellic acid; Centaurium extract, nordihydroguaiaretic acid; ginger oil; retinol; Polygonum Cuspidatum extract; cedar himalaya extract; tetrahydrocurcuminoids; Nigella Sativa essential oil; copaiba balsam; Rhodiola Rosea; frankincense; blue cypress oil; blue chamomile oil; vetiver mada; bulgarian rose oil; resveratrol ferulate; Helichrysum oil; rosmarinic acid; resveratrol triphosphate, noni leaf extract; stromelysin-3; endrin; glucocorticoids; Caralluma Fimbriata Extract, Mucuna Extract, Bacana extract, forskolin; nicotine; deoxycholate; theophylline; caffeine; theobromine; magnolol (a fraction of Magnolia extract); isoproterenol; kaempferol-7-O-Neohesperidoside; Clary extract; Chrystanthellum extract, specifically Chrysanthellum Indicum; and the like.

15. A kit for modulating the appearance of volume on select facial or body surfaces in need thereof comprising:
(a) at least one agent for increasing the appearance of volume when used to treat a selected keratin surface;
(b) at least one agent for decreasing the appearance of volume when used to treat a selected keratin surface.

16. The kit of claim 15 wherein the at least one agent (a) is a topically applied composition contained in a receptacle.

17. The kit of claim 15 wherein the at least one agent (b) is a topically applied composition contained in a receptacle.

18. The kit of claim 15 wherein the agents (a) and (b) are in the form of a skin cream or lotion.

19. The kit of claim 15 wherein the agents (a) or (b) are in the form of a color cosmetic composition.

20. The kit of claim 15 additionally comprising a third component which is a moisturizer or facial makeup composition.
